(19) United States
(12) Patent Application Publication
(10) Pub. No.: US 2026/0137196 A1
(43) Pub. Date: May 21, 2026

(54) METHOD AND SYSTEM OF MANUFACTURING PERSONALIZED MASK-PACK

(71) Applicant: SKINDAUM CO., LTD, Gyeonggi-do (KR)

(72) Inventor: Young Kyoung Lee, Seoul (KR)

(21) Appl. No.: 19/106,159

(22) PCT Filed: Jul. 19, 2023
(86) PCT No.: PCT/KR2023/010351

§ 371 Date: Jan. 6, 2026

(30) Foreign Application Priority Data
Aug. 26, 2022 (KR) 10-2022-0107950

(51) Int. Cl. A45D 44/00; A61B 5/00

(52) U.S. Cl. CPC A45D 44/002; A61B 5/0077; A61B 5/441; A45D 2044/007

(57) ABSTRACT

A system for manufacturing a customized mask pack comprises a user terminal generating first skin measurement information and skin input information including an image of the user's face; at least one IoT beauty device being associated with the user terminal and generating the user's second skin measurement information; a database storing big data including training data for predicting skin diagnosis information of the user from the first and second skin measurement information and the skin input information; an artificial intelligence (AI) server extracting patterns from the first and second skin measurement information and the skin input information based on the big data, and predicting the skin diagnosis information of the user from the extracted patterns; a home beauty mask pack providing device generating output data of the user corresponding to the predicted skin diagnosis information.

### TECHNICAL FIELD

Embodiments of the present disclosure relate to electronic devices, methods for manufacturing the same, and devices including electronic devices, and more particularly, to transistors, methods for manufacturing the same, and semiconductor devices/devices including transistors.

### BACKGROUND ART

As human skin ages, it undergoes physicochemical changes such as loss of skin elasticity, formation of skin wrinkles, changes in skin color, loss of skin barrier function, and loss of skin immune function. In particular, stimulating substances in the external environment such as heavy metals, yellow dust, ozone, ultraviolet rays, cosmetic ingredients, etc. promote the decline in the activity of skin cells, necrosis of skin cells, and cell degeneration, which promotes wrinkle formation, pigmentation, loss of immune function, and increased inflammation. To prevent and improve these conditions, beauty mask packs are used, and conventional beauty mask packs have been manufactured with essences containing nutritional substances such as natural extracts, proteins, vitamins, etc. on woven or non-woven fabrics to functions such as whitening, anti-wrinkle, moisturizing, relieving skin problems, and providing skin elasticity.

However, despite the fact that each person's skin color, degree of aging, sensitivity, genetic factors, etc. vary widely, and have different positions and areas of eyes, nose, and mouth, conventional mask packs are not optimized for consumers, but are manufactured by producers, so the effectiveness of the mask pack varies, and the mask pack does not accurately reflect the size of the user's face, and the active ingredients are distributed throughout, so the effect cannot be applied to the desired local area, and the effectiveness is reduced due to the loss of active ingredients.

Recently, the home beauty device market has been booming as the number of ‘Home beauty people’ who take care of their skin and hair at home has increased. As the novel coronavirus (COVID-19) pandemic continues, more consumers are looking for easy and convenient care at home using home beauty devices instead of visiting skin care facilities or dermatologists. In addition, beauty-related products, which used to be limited to LED masks and cleansing devices, have been gradually subdivided, and various skin that combine with IT technology have been launched in response to this market situation.

However, because these home beauty devices are based on passive input from users, they are limited in their ability to provide truly personalized beauty services for various skin concerns, such as by area and type.

### DISCLOSURE OF THE INVENTION

### Technical Problem

The technological object to be achieved by embodiments of the preset disclosure is to provide a personalized face mask manufacturing system based on home beauty.

In addition, the technological object to be achieved by embodiments of the present disclosure is to provide a personalized face mask manufacturing system that is simple, convenient, economical, efficient, and capable of precise skin diagnosis.

In addition, the other technological object to be achieved by embodiments of the present disclosure is to provide a method for manufacturing a customized mask pack having the aforementioned advantages.

### Technical Solution

According to one embodiment of the present disclosure, a system for manufacturing a customized mask pack comprises a user terminal generating first skin measurement information and skin input information including an image of the user's face; at least one IoT beauty device being associated with the user terminal and generating the user's second skin measurement information; a database storing big data including training data for predicting skin diagnosis information of the user from the first and second skin measurement information and the skin input information; an artificial intelligence (AI) server extracting patterns from the first and second skin measurement information and the skin input information based on the big data, and predicting the skin diagnosis information of the user from the extracted patterns; a home beauty mask pack providing device generating output data of the user corresponding to the predicted skin diagnosis information, and providing a customized mask pack based on the output data of the user.

In one embodiment, the user terminal or the IoT beauty device generates a third skin measurement information, after the customized mask pack is used by the user.

The user terminal generates a first facial image of the user based on the first and second skin measurement information, generates a second facial image of the user based on the third skin measurement information; and displays a comparison of the first facial image and the second facial image.

The user terminal receives the predicted skin diagnosis information from the artificial intelligence server, generates a virtual mask pack based on the predicted skin diagnosis information, superimposes the virtual mask pack on a face image of the user to display an output image, and displays an animated effect of skin being improved by the virtual mask pack on the output image.

The AI server divides the user's facial image into a plurality of sub-regions, forms a plurality of partial facial images corresponding to the plurality of sub-regions, and generates the skin diagnostic information for each facial region of the user based on the plurality of partial facial images, and a resolution of the face image of the user is the same as the resolution of the partial face image.

The skin diagnostic information comprises the elasticity, moisture, pigmentation, antioxidants, and sensitivity of skin, and the elasticity, moisturization, pigmentation, antioxidant, and sensitivity of the skin is read from data including pores, skin temperature, oil, moisture, skin tone, skin pH, skin thickness, wrinkles, pigment, blemishes, sebum, and dead skin cells of the user, and the data is predicted from the first and second skin measurement information and the skin input information.

The skin input information comprises at least one of: lifestyle information extracted from a social networking service (SNS) of the user, the skin input information being information about a skin diagnostic questionnaire completed by the user, location information of the user received from a GPS receiver, skin disease information of the user, genetic information of the user, and humidity and temperature of a location where the user is located.

The skin measurement information comprises at least one of: the degree of UV exposure to which the user is exposed, the oiliness, moisture, skin temperature, skin pH, and skin thickness of the user's skin, and the customized mask pack comprises any one of a U-zone, T-zone, nose pack, eye pack, wrinkle pack, neck pack, and face mask.

According to another embodiment of the present disclosure, an apparatus for providing a home beauty mask pack comprises a communication part receiving the user's predicted skin diagnosis information; a control unit for generating output data from the user corresponding to the predicted skin diagnosis information; and a supply unit providing a customized mask pack for the user based on output data of the user, wherein the supply unit includes a first supply portion for supplying a hydrogel and a second supply portion for supplying a mask pack ingredient, and wherein the skin diagnosis information of the user is determined from patterns extracted from the first and second skin measurement information and the skin input information based on big data, and, wherein the first skin measurement information includes a facial image of the user, and is generated via the user terminal with the skin input information, and wherein the second skin measurement information is generated via at least one IoT beauty device linked to the user terminal, and, wherein the skin diagnosis information of the user is predicted by learning from the first and second skin measurement information and the skin input information.

In addition, according to another embodiment of the present disclosure, a method of manufacturing a customized mask pack comprises generating a first skin measurement information and a skin input information including a face image of a user; generating a second skin measurement information of the user by being linked a user terminal to at least one IoT beauty device; storing big data including learning data to predict skin diagnosis information of the user from the first and second measurement information and the skin input information in a database; extracting patterns from the first and second skin measurement information and the skin input information based on the big data in an artificial intelligence server; predicting the skin diagnosis information of the user based on the patterns; generating output data of the user corresponding to the skin diagnosis information by receiving the skin diagnosis information in a home beauty mask pack providing apparatus; and providing a customized mask pack for the user based on the output data of the user.

### Advantageous Effects

According to embodiments of the present disclosure, by generating and storing big data based on various user skin input information and skin measurement information obtained from a user terminal and an IoT beauty device, and predicting a user's skin diagnosis through an artificial intelligence algorithm based on the big data, a home beauty mask pack providing device provides a customized mask pack based on the predicted skin diagnosis information, a personalized mask pack manufacturing system capable of precise skin diagnosis with simplicity, convenience, economy, and efficiency can be provided.

Further, according to another embodiment of the present disclosure, a method of manufacturing a customized mask pack having the above advantages may be provided.

### BRIEF DESCRIPTION OF THE DRAWINGS

FIG. 1 illustrates a customizable mask pack manufacturing system according to embodiments of the present disclosure.

FIG. 2 is a block diagram of a home beauty mask pack delivery device according to an embodiment of the present disclosure.

FIG. 3 is an example illustrating of dividing an image of a user's face into sub-regions, according to an embodiment of the present disclosure.

FIGS. 4A to 4F illustrate types of mask packs according to embodiments of the present disclosure.

FIG. 5A illustrates an example of a comparison of a user's face before and after using a mask pack according to an embodiment of the present disclosure, and FIG. 5B illustrates a future user's face that would appear if the user left the skin untreated for an extended period of time.

FIG. 6 illustrates a method of operation of a customizable mask pack manufacturing system according to embodiments of the present disclosure.

FIG. 7 illustrates a method of operation of a user terminal in a customized mask pack manufacturing system according to an embodiment of the present disclosure.

### BEST MODE FOR CARRYING OUT THE INVENTION

Hereinafter, the embodiments of the present disclosure will be described in detail with reference to the accompanying drawings.

The embodiments of the present disclosure below are provided to more fully illustrate the invention to one of ordinary skill in the art, and the following embodiments may be modified in many other ways, and the scope of the invention is not limited to the following embodiments. Rather, these embodiments are provided to make the invention more complete and complete and to fully convey the ideas of this invention to those skilled in the art.

Further, in the following drawings, the thickness or size of each layer is exaggerated for convenience and clarity of description, and like designations in the drawings refer to like elements. As used herein, the term “and/or” includes any of the enumerated items and any combination of one or more of the enumerated items.

The terms used herein are intended to describe specific embodiments and are not intended to limit the invention. As used herein, the singular form may the form, unless the context clearly indicates otherwise. Furthermore, as used herein, the words “comprise” and/or “comprising” are intended to specify the presence of the mentioned shapes, numbers, steps, actions, members, elements, and/or groups thereof, and not to exclude the presence or addition of one or more other shapes, numbers, actions, members, elements, and/or groups thereof. Terms such as first, second, and the like may be used to describe various components, but the components are not to be limited by such terms. The terms are used only for the purpose of distinguishing one component from another.

Embodiments of the present invention will hereinafter be described with reference to the drawings, which schematically illustrate idealized embodiments of the present invention. In the drawings, the sizes and shapes of members, for example, may be exaggerated for convenience and clarity of description, and variations in the shapes shown may be expected in actual implementation. Accordingly, embodiments of the present invention should not be construed as limited to the specific geometries of the members or areas shown herein. Various embodiments of the present invention will be described hereinafter with reference to the drawings, which are available at.

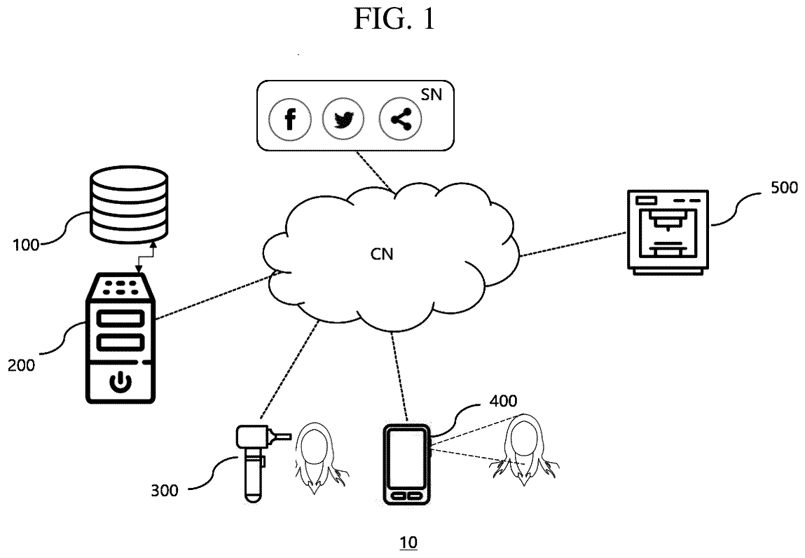

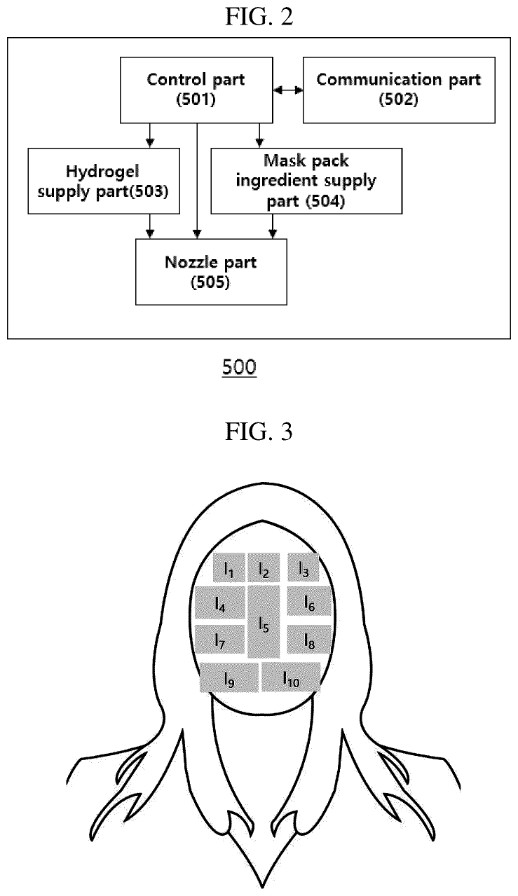

FIG. 1 illustrates a customizable mask pack manufacturing system 10 according to an embodiment of the present disclosure, and FIG. 2 is a block diagram of a home beauty mask pack delivery device according to an embodiment of the present disclosure.

Referring to FIG. 1, a user-customized mask pack manufacturing system 10 may include a database 100, an artificial intelligence server 200, an IoT beauty device 300, a user terminal 400, and a home beauty mask pack delivery device 500. The database 100, the artificial intelligence server 200, the IoT beauty device 300, the user terminal 400, and the home beauty mask pack delivery device 500 may transmit and receive data to and from each other via the communication network CN. The communication network CN may comprise a wired communication network, a mobile communication network, a wireless network, and combinations thereof. For example, the wired communication network may include an Internet Protocol (IP)-based wired network. The cellular network includes a 5G network, a long term evolution (LTE) network, or a wideband code division multiple access (WCDMA) network. The wireless network includes various types of wireless networks, such as Wi-Fi networks. However, the communication network CN is not to a particular technology, as it may both established as described above as well as networks developed in the future. The user terminal 400 may be a smartphone, tablet personal computer, mobile phone, video phone, e-book reader, desktop personal computer, laptop personal computer, netbook computer, workstation, server, personal digital assistant (PDA), portable multimedia player (PMP), MP3 player, mobile medical device, wearable device (e.g., smart glasses, head-worn device, etc: smart glasses, head-mounted-device (HMD)), and may include at least one of the following.

The database 100 may include a plurality of storage devices and may store various data. In an embodiment of the present invention, the database 100 may store information related to a user's skin diagnosis and data for training the AI. The skin diagnosis-related information may be generated by the user terminal 400 or the IoT beauty device 300, and the data for AI training may collectively refer to data utilized for training AI models such as machine learning and deep learning. The IoT beauty device 300 may include skin measurement sensors, such as pH measurement sensors, moisture measurement sensors, and oil measurement sensors, and may generate skin measurement signals of the user, such as pH data, moisture data, and oil data.

The artificial intelligence server 200 may accumulate the data input via the database 100 in the AI platform into time series, and predict the skin diagnosis of the user based on the accumulated time series data and the skin diagnosis prediction model. In one embodiment, the prediction of the skin diagnosis of the user may be performed by unsupervised learning that looks for inherent patterns in the data using skin measurement information and skin input information as inputs. Machine learning can be defined as a methodology for empowering computers to learn on their own without being explicitly programmed, in which a program learns patterns in data by itself based on data. Machine learning is divided into supervised learning and unsupervised learning depending on whether the correct answer is specified in the data required for learning, and is divided into classification (Classification), which divides data into finite categories according to the purpose of use, regression, which maps to continuous values, clustering, which groups similar data, and dimension reduction methodology, which reduces multidimensional data to a representative lower dimension.

Deep learning is one of the machine learning techniques and is a methodology based on the Artificial Neural Network (ANN) algorithm that the of in the human brain structure, and the deep structure is a deep neural network (DNN) that multiple hidden layers between the input layer and the output layer, a convolutional neural network (CNN) that learns to extract factors in front of the hidden layer, and a deep neural network (DNN) that learns to process time series data: Deep Neural Network (DNN), which has multiple hidden layers between the Input Layer and the Output Layer; Convolutional Neural Network (CNN), which has filters required for factor extraction before the hidden layer and learns the filters together; and Recurrent Neural Network (RNN), which can process time series data by stacking artificial neural networks at each time. The high performance of deep learning models is explained by two things: first, artificial neural networks are universal approximators that may approximate any kind of function as a superposition of weighted sums of functions in each layer, which means that they can simulate data with high accuracy given sufficiently general data. Second, it is important to properly extract the factors that represent the data in order to classify the data well, and the spiral neural network can be used to extract the optimal factors through filter learning. In addition, deep learning is an evolution of a model in the field of artificial intelligence called neural network, which is a hierarchical structure in which the hidden layer of an artificial neural network is composed of multiple levels. Recent deep learning models have many internal layers, and the number of weights (meaning connection strength) connecting nodes can be up to billions. The above convolutional neural network (CNN) is a model that mimics the visual processing of living things and has the advantage of patterns when the size or position of the pattern changes. It has features: local receptive fields, shared weights, and subsampling. First, a local receptive field means that each node in the lower layer is connected by a weight (filter) to only a subset of nodes in the upper layer, as opposed to the traditional model where each node in the lower layer is connected to all nodes in the upper layer. When a filter is applied to all regions of an input pattern and the weights are multiplied by those regions to sum them, passing them to the nodes in the panel at the parent node is called a convolution. Next, weight sharing means that the weights are shared, meaning that the nodes in one panel and the filters applied to the input pattern (or sublayer) are the same. Since one filter can recognize one feature (or small pattern), one panel will be able to find the corresponding feature present at a specific location in the input pattern. Third, is a that occurs the second third layers to the panels present in the second layer, also known as pooling. In this case, Average Pooling, which averages and multiplies the weights, is often used, but in one embodiment of the present invention, Max Pooling, which uses the largest of the four values in the 2×2 region, is used. has the function of the size of the pattern and making it less sensitive to in the location of the found features. This process of convolution and may be multiple times according to embodiments, and after is, the neural network is connected.

When training the weights of the convolutional neural network, the convolutional neural network has a large number of inner layers, so the number of nodes becomes large and the weights connecting the nodes become large. In general, when the number of weights in a model increases, the error backpropagation algorithm will cause the size of the first-order likelihood function to become very large or even zero in the process of repeatedly calculating it. However, in the case of convolutional neural networks, the number of weights is not large enough to cause such a problem, so the error backpropagation algorithm can be applied without difficulty. However, the shared weights feature and the process of are from typical neural network models, so the algorithm can be modified. For example, for shared weights, you can calculate the first-order likelihood function in the same way as for different weights, and then add all the values of the first-order likelihood function for the same weights. If the pattern is, it is necessary to perform when propagating the error, process of by up-sampling may be added. However, it will be apparent that various methods can be utilized without being limited to the above methods. The recurrent neural network (RNN) model is suitable for processing sequential information such as speech recognition or language recognition. RNNs receive and process the elements that make up sequential data one by one, and store the processed information in internal nodes, which are created according to the input time, store the information processed up to that point, and output it through output nodes as needed.

In addition, the recurrent neural network model may be applied to various applications depending on the type of data input and output, including but not limited to machine translation or annotation of videos when the input and output data are sequential, sentiment analysis when the output is non-sequential, and annotation of photos when the input is non-sequential. Finally, deep belief network is an unsupervised learning model, which is characterized by the fact that humans or animals learn by themselves through observation. It will be appreciated that various machine learning methods may be utilized, including but not limited to those listed, and may be modified according to embodiments.

In one embodiment, the user terminal 400 may generate first skin measurement information and skin input information comprising a facial image of the user. Specifically, the user terminal 400 may generate the first skin measurement information by photographing the user via an internal camera module. The skin input information may be obtained via a social networking service (SNS), a skin diagnostic questionnaire, and a GPS receiver, and may include at least one of lifestyle information extracted from a social networking service (SNS) of the user, information about an electronic skin diagnostic questionnaire completed by the user, location information of the user received from the GPS receiver, information about a skin disease of the user and genetic information of the user issued by a specialized organization, and humidity, temperature, particulate matter, ultraviolet radiation, and ozone index of a place where the user is located from a site providing weather information. The social networking service (SN) may any of Facebook, Twitter, YouTube, Instagram, TikTok, KakaoTalk, Pinterest, LinkedIn, Band, KakaoGroup, Between, Polar. The lifestyle information may include at least one of the following: the user's dietary habits, hydration, outdoor activities, diet, pregnancy/birth, menstruation, sleep deprivation, seasonal changes, and stress.

The IoT beauty device 300 may be coupled to a user terminal 400 and may generate second skin measurement information of the user. The second skin measurement information is a value measured via various sensors and may include at least one of the following: the degree of UV exposure to which the user is exposed, the oiliness, moisture, skin temperature, skin pH, and skin thickness of the user's skin. The user terminal 400 may receive the second skin measurement information from the IoT beauty device 300 and display it visually to show it to the user.

The database 100 may store big data comprising training data for predicting skin diagnosis information of the user from the first and second skin measurement information and the skin input information. The artificial intelligence server 200 may extract certain patterns from the first and second skin measurement information and the skin input information based on the big data, and predict skin diagnosis information of the user from the extracted certain patterns. The skin diagnostic information may include a degree of elasticity, moisture, pigment, antioxidant, and sensitivity of the skin, and the elasticity, moisture, pigment, antioxidant, and sensitivity of the skin may be read from data including pores, skin temperature, oil, moisture, skin tone, skin pH, skin thickness, wrinkles, pigment, blemishes, sebum, and keratin of the user, and the data may be predicted from the first and second skin measurement information and the skin input information.

The home beauty mask pack providing device 500 may receive the predicted skin diagnosis information from, generate output data corresponding to the predicted skin diagnosis information, and provide a customized mask pack for the user based on the output data. The output data may comprise a StereoLithography (STL) file. In another embodiment, the output data may be generated by the artificial intelligence server 200 and provided to the home beauty mask pack delivery device 500.

The home beauty mask pack providing apparatus 500 may include a plurality of storage members for receiving a plurality of cosmetic ingredients, and a plurality of nozzle members for dispensing the cosmetic ingredients from the storage members onto the prepared mask pack sheet, or for providing the cosmetic ingredients to a space in the prepared mask pack for absorption of the cosmetic ingredients within the mask pack. The home beauty mask pack providing apparatus 500 may further include a receiving member for storing each of the various shapes of mask pack sheets (e.g., T-zone, U-zone, V-zone, nose pack, eye pack, etc.) to be described later. The mask pack sheets stored in the receiving member may be selected according to the predicted skin diagnosis information and one of them may be output to a space in which a cosmetic ingredient is supplied, and the cosmetic ingredient may be dispersed or supplied on the interior and surface of the output mask pack sheet according to the predicted skin diagnosis information.

The cosmetic ingredients may be whitening, anti-wrinkle, nourishing, moisturizing, allergy care, and exfoliating ingredients, each of which may be stored in a respective storage member. The cosmetic ingredient may be stored in liquid, powder, capsule, pill, or mixture thereof.

The home beauty mask pack delivery device 500 may be operable by voice recognition and, as an additional feature, may have a cold and hot function to cool or warm the mask pack sheet on which the active ingredients are dispersed or absorbed. For example, a warm mask pack may be used in cold weather, such as winter, and a cold mask pack can be used in hot weather to improve user comfort.

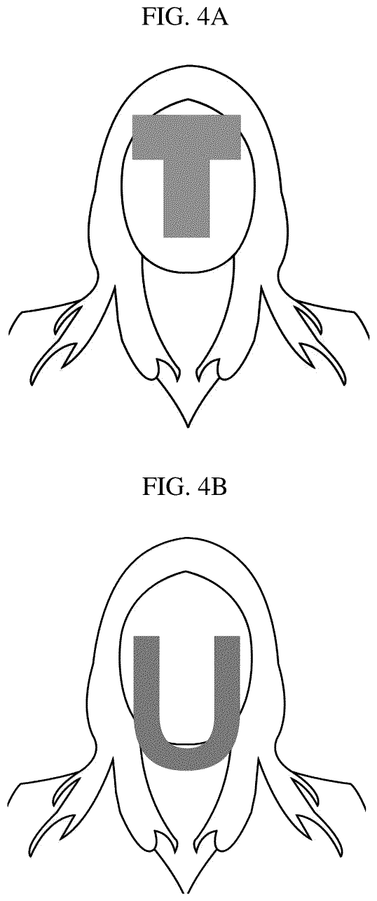

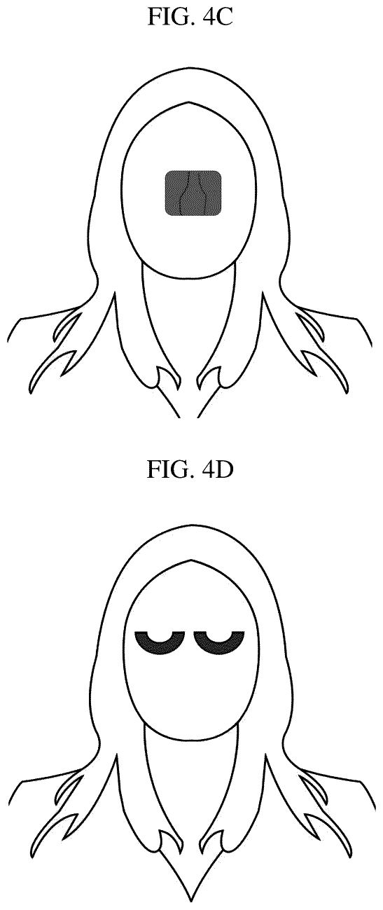

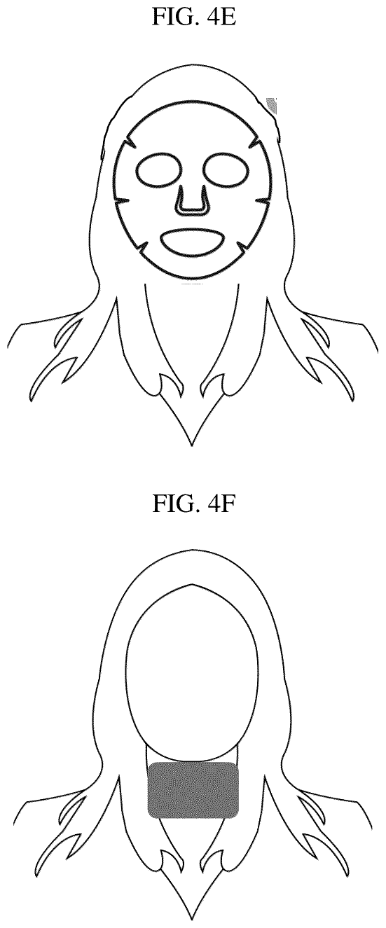

In another embodiment, referring to FIG. 2, the home beauty mask pack delivery device 500 may comprise a control part 501, a communication part 502, a hydrogel supply part 503, a mask pack ingredient supply part 504, and a nozzle part 505. The communication part 502 may receive the skin diagnosis information from the AI server 200 and provide it to the control part 501. The control part 501 controls the overall home beauty mask pack delivery device 500, and may adjust the amount of hydrogel stored in the hydrogel supply part 503 and impregnant stored in the mask pack ingredient supply part 504 provided to the nozzle part 505 based on the above skin diagnosis information received from the communication part 502. By adjusting the amount of hydrogel and the amount of impregnating liquid based on the skin diagnosis information of the user, a customized mask pack for the user may be provided. For example, based on the skin diagnosis information, the addition of lipophilic ingredients and the amount of efficacy ingredients (e.g., ingredients related to whitening, moisturizing, firming, wrinkle, acne, soothing, sebum control, and pore control and pore reduction) may be determined. The hydrogel supply part 503 may supply the hydrogel material to the nozzle part 505 based on the skin diagnosis information of the user under the control of the control part 501, and the mask pack ingredient supply part 504 may supply the impregnating liquid to the nozzle part 505 based on the skin diagnosis information of the user under the control of the control part 501. The nozzle part 505 may spray the materials stored in the hydrogel supply part 503 and the mask pack ingredient supply part 504 onto a plate (not shown) according to a 3D printing control command for manufacturing a mask pack comprising a preset 3D printed hydrogel sheet to print a mask pack comprising a 3D printed hydrogel sheet. The nozzle portion 505 may further comprise a heating element (not shown) that controls the temperature of the nozzle portion between 50 and 120° C. to prevent the injected hydrogel from hardening and solidifying in the nozzle. The printed mask pack may be provided in various forms, such as FIGS. 4a to 4f, which will be described later, based on the skin diagnostic information.

In one embodiment, after the user uses the customized mask pack, the user may generate third skin measurement information using the user terminal 400 or IoT beauty device 300. Specifically, the third skin measurement information is the user's skin measurement information that is measured after the user has used the customized mask pack, and may be compared to the second skin measurement information prior to using the mask pack to allow the user to visually see skin improvement information. Specifically, the user terminal 400 may generate a first face image of the user based on the first and second skin measurement information, generate a second face image of the user based on the third skin measurement information, and display the first face image and the second face image by comparing the first face image and the second face image.

Further, the user terminal 400 may receive the predicted skin diagnosis information from the artificial intelligence server 200, generate a virtual mask pack based on the predicted skin diagnosis information, and display an output image by superimposing the virtual mask pack on a face image of the user. The facial image of the user may be an image taken and stored via a camera. Wherein, an animated effect of the skin being improved by the virtual mask pack is displayed on the output image, thereby allowing the user to visually see the extent to which the user's skin is improved when using the customized mask pack.

As described above, the mask pack manufacturing system 10 may diagnose the skin condition of a user through a skin application of a smart device that through an authentication procedure, measures the oil and requires by the user, and supplies materials containing ingredients required for the skin, and a customized mask on artificial intelligence for each area. For example, if the user has dry eyes, oily forehead, oily mouth, and dry cheeks, the eye patch is for dry eyes, the forehead is for oily forehead, and other areas are for dry cheeks, the user may make and use a mask pack at home with ingredients that match the skin type of each area.

FIG. 3 is an example illustrating of dividing an image of a user's face into sub-regions, according to an embodiment of the present disclosure.

Referring to FIG. 3, the AI server 200 may divide the face image of the user into a plurality of sub-regions I1 to I10, and form a plurality of partial face images corresponding to the plurality of sub-regions I1 to I10. The plurality of sub-regions (I1, I2, I3, I5) are associated with a T-zone, wherein the T-zone includes the area around the glabella and the tip of the nose. The plurality of subregions (I7, I9, I10, I8) are associated with the U-zone, wherein the U-zone includes the periphery of the bilateral nasolabial folds. The plurality of subregions I4, I6 include the area. The plurality of sub-zones in FIG. 3 is an example, and may be further subdivided and distinguished, and the invention is not limited thereto. Based on the plurality of partial face images, skin diagnostic information may be generated for each region of the user's face. Thus, by segmenting the face of the user, the skin diagnosis may be more precise and accurate in environments where the skin characteristics of different parts of the face are different. By matching the resolution of the user's face image with the resolution of the partial face image, the accuracy of the skin diagnosis may be further maintained.

FIGS. 4A to 4F illustrate types of mask packs according to embodiments of the present disclosure. The aforementioned user-customizable mask pack shapes may include any one of T-zone, U-zone, nose pack, eye pack, eye wrinkle pack, neck pack, and face mask shapes.

FIG. 4A shows that a customized mask pack in the shape of the T-zone is provided based on the skin diagnosis information above and is worn on the face of the user, and FIG. 4B shows that a customized mask pack in the form of a U-zone is provided based on the above skin diagnosis information and is worn on the face of the user, FIG. 4D shows that a customized mask pack in the form of a nose pack is provided based on the above skin diagnosis information and is worn on the face of the user, and FIG. 4D shows that a customized mask pack in the form of an eye patch is provided based on the above skin diagnosis information and is worn under the eyes of the user. FIG. 4E shows that a customized mask pack in the form of a mask that covers the entire face is provided based on the above skin diagnosis information and is worn over the entire face of the user, and FIG. 4F shows that a customized mask pack in the form of a mask that covers a portion of the neck is provided based on the above skin diagnosis information and is worn on the neck of the user.

As described above, user may perform a skin diagnosis of the user based on the AI platform through his skin input information and skin measurement information, and use a pack based on the skin diagnosis result, thereby providing a reliable customized pack to the user.

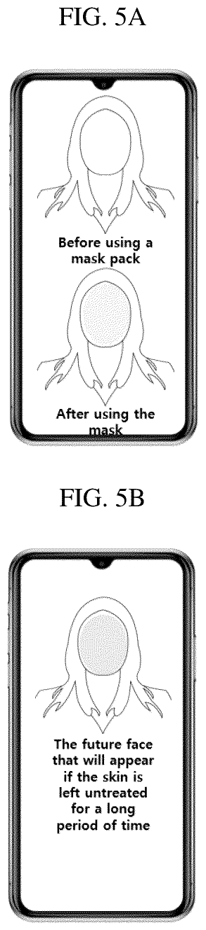

FIG. 5A illustrates an example of a comparison of a user's face before and after using a mask pack according to an embodiment of the present disclosure, and FIG. 5B is a drawing of a future user's face that would appear if the user left the skin untreated for an extended period of time.

Referring to FIG. 5A, the display of the user terminal 400 may display a first user image before use of the customized mask pack and a second use image after use. This allows the user to visually see how much their skin has by comparing the before and after conditions of the customized mask pack, which can be to the user.

Referring to FIG. 5B, a virtual representation of the future skin and facial appearance of a user may be shown if the user leaves their skin untreated for an extended period of time. By providing a hypothetical image of the user when the skin is left untreated for a long period of time, the user may be made aware of the need for skin care so that the user can continue to take care of themselves.

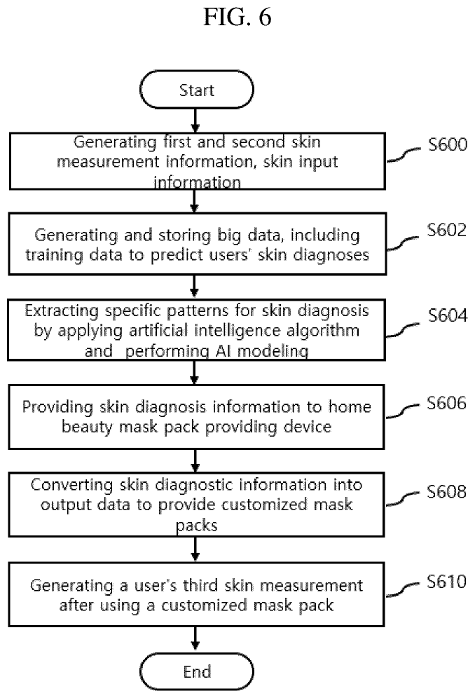

FIG. 6 illustrates a method of operation of a customizable mask pack manufacturing system according to embodiments of the present disclosure.

Referring to FIG. 6, a home beauty mask system (S600), wherein a user terminal generates first skin measurement information and skin input information comprising a facial image of a user, and at least one IoT beauty device is associated with the user terminal and generates second skin measurement information of the user, wherein a database stores big data comprising training data for predicting skin diagnosis information of the user from the first and second skin measurement information and the skin input information (S602). A method for manufacturing a customized mask pack for a user may be provided, wherein the artificial intelligence server extracts a specific pattern from the first and second skin measurement information and the skin input information based on the big data, and predicts a skin diagnosis information of the user from the extracted specific pattern (S604), wherein a home beauty mask pack providing device receives the predicted skin diagnosis information (S606), and generates output data of the user corresponding to the predicted skin diagnosis information, and provides a customized mask pack for the user based on the output data of the user (S608). Optionally, after the user uses the customized mask pack, the user terminal or the IoT beauty device may generate a third skin measurement information. By comparing the customized mask pack after use with a pre-use condition, the user may visually see the extent to which their skin is improved, which may increase user satisfaction.

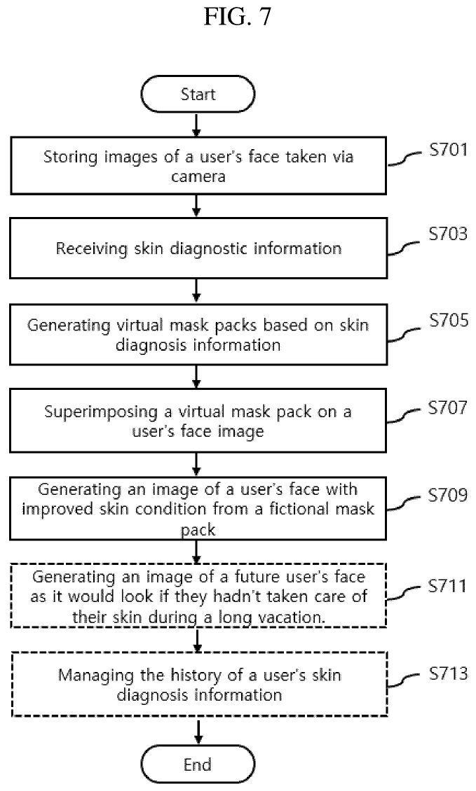

FIG. 7 illustrates a method of operation of a user terminal in a customized mask pack manufacturing system according to an embodiment of the present disclosure.

Referring to FIG. 7, the user terminal may include: generating and storing a selfie face image of a user via an internal camera module (S701); receiving a predicted skin diagnosis information of the user from an artificial intelligence server (S703); generating a virtual mask pack based on the predicted skin diagnosis information (S705), displaying an output image by superimposing the virtual mask pack on a face image of the user (S707), and displaying an animation effect in which the skin is improved by the virtual mask pack (S709), a method of operating a user terminal may be provided. The face image of the user may be obtained by accessing a storage space, such as a cloud server, and downloading a previously stored photograph of the user.

Optionally, the user terminal may further comprise a step (S711), as shown in FIG. 5B, of virtually showing the future skin and facial appearance that would appear if the user left the skin untreated for a long period of time. By providing a virtual image of the user when the skin has not been cared for a long period of time, the user may be made aware of the need to care for the skin so that the user can continue to care for the skin on their own. The user terminal may include a step (S713) of managing a history of the user's skin diagnosis information. By managing a history of the user's skin diagnosis information, the user may view changes in the user's skin condition, which may provide a sense of satisfaction of skin improvement or motivation for skin care. Further, the user terminal may include the steps of generating a first facial image of the user based on the first and second skin measurement information, generating a second facial image of the user based on the third skin measurement information, and displaying a comparison of the first facial image and the second facial image, as shown in FIG. 5A.

As described above the present does not rely on user inputs such as selfie photos taken by users for skin diagnosis, but utilizes data measured from various IoT skin measurement together, enabling precise skin diagnosis. In addition, in line with the recent trend of pursuing a free lifestyle at home, the invention may be used in conjunction with a smartphone app to manufacture and use various mask packs that are subdivided by skin based on the user's skin condition using skin measurement and artificial intelligence algorithms, thereby increasing the accessibility, efficiency, and convenience of users.

In addition, through continuous skin diagnosis analysis, it is possible to provide personalized skin care ingredients in real time, formulate the optimal required ingredients for each facial area (T-zone, U-zone, EYE, CHEEK, etc.), and create customized patches (eye, cheek, forehead, neck, face) by shape as well as function at home, and provide scientific skincare solutions that address skin changes and future skin concerns through skin analysis history.

In addition, the present invention has simplicity, convenience, economy, and efficiency by precisely diagnosing what and how much nutritional ingredients are needed for each part of the user's face, continuously monitoring and managing the user's skin condition, and enabling the user to make and use mask packs for specific parts of the face at home by himself or herself, according to the individual's skin condition and weather, humidity, season, living area, personal schedule, and taste.

It will be apparent to one of ordinary skill in the art to which this invention belongs that the invention described above is not limited to the foregoing embodiments and accompanying drawings, and that various substitutions, modifications, and changes are possible without departing from the technical ideas of the invention.

1. A system for manufacturing a customized mask pack comprising:
a user terminal generating first skin measurement information and skin input information including an image of the user's face;
at least one IoT beauty device being associated with the user terminal and generating the user's second skin measurement information;
a database storing big data including training data for predicting skin diagnosis information of the user from the first and second skin measurement information and the skin input information;
an artificial intelligence (AI) server extracting patterns from the first and second skin measurement information and the skin input information based on the big data, and predicting the skin diagnosis information of the user from the extracted patterns;
a home beauty mask pack providing device receiving the predicted skin diagnosis information, generating output data of the user corresponding to the predicted skin diagnosis information, and providing a customized mask pack for the user based on the output data of the user.

2. The system of claim 1, wherein the user terminal or the IoT beauty device generates a third skin measurement information, after the customized mask pack is used by the user.

3. The system of claim 2, wherein the user terminal generates a first facial image of the user based on the first and second skin measurement information, generates a second facial image of the user based on the third skin measurement information; and displays a comparison of the first facial image and the second facial image.

4. The system of claim 1, wherein the user terminal receives the predicted skin diagnosis information from the artificial intelligence server, generates a virtual mask pack based on the predicted skin diagnosis information, superimposes the virtual mask pack on a face image of the user to display an output image, and displays an animated effect of skin being improved by the virtual mask pack on the output image.

5. The system of claim 1,
wherein the AI server divides the user's facial image into a plurality of sub-regions, forms a plurality of partial facial images corresponding to the plurality of sub-regions, and generates the skin diagnostic information for each facial region of the user based on the plurality of partial facial images,
wherein a resolution of the face image of the user is the same as the resolution of the partial face image.

6. The system of claim 1,
wherein the skin diagnostic information comprises the elasticity, moisture, pigmentation, antioxidants, and sensitivity of skin,
wherein the elasticity, moisturization, pigmentation, antioxidant, and sensitivity of the skin is read from data including pores, skin temperature, oil, moisture, skin tone, skin pH, skin thickness, wrinkles, pigment, blemishes, sebum, and dead skin cells of the user,
wherein the data is predicted from the first and second skin measurement information and the skin input information.

7. The system of claim 1, wherein the skin input information comprises at least one of: lifestyle information extracted from a social networking service (SNS) of the user, the skin input information being information about a skin diagnostic questionnaire completed by the user, location information of the user received from a GPS receiver, skin disease information of the user, genetic information of the user, and humidity and temperature of a location where the user is located.

8. The system of claim 1, wherein the skin measurement information comprises at least one of: the degree of UV exposure to which the user is exposed, the oiliness, moisture, skin temperature, skin pH, and skin thickness of the user's skin.

9. The system of claim 1, wherein the customized mask pack comprises any one of a U-zone, T-zone, nose pack, eye pack, wrinkle pack, neck pack, and face mask.

10. An apparatus for providing a home beauty mask pack comprising:
a communication part receiving the user's predicted skin diagnosis information;
a control part for generating output data from the user corresponding to the predicted skin diagnosis information; and
a supply unit providing a customized mask pack for the user based on output data of the user,
wherein the supply unit includes a first supply portion for supplying a hydrogel and a second supply portion for supplying a mask pack ingredient,
wherein the skin diagnosis information of the user is determined from patterns extracted from the first and second skin measurement information and the skin input information based on big data,
wherein the first skin measurement information includes a facial image of the user, and is generated via the user terminal with the skin input information,
wherein the second skin measurement information is generated via at least one IoT beauty device linked to the user terminal,
wherein the skin diagnosis information of the user is predicted by learning from the first and second skin measurement information and the skin input information.

11. A method of manufacturing a customized mask pack comprising:
generating a first skin measurement information and a skin input information including a face image of a user;
generating a second skin measurement information of the user by being linked a user terminal to at least one IoT beauty device;
storing big data including learning data to predict skin diagnosis information of the user from the first and second measurement information and the skin input information in a database;
extracting patterns from the first and second skin measurement information and the skin input information based on the big data in an artificial intelligence server;
predicting the skin diagnosis information of the user based on the patterns;
generating output data of the user corresponding to the skin diagnosis information by receiving the skin diagnosis information in a home beauty mask pack providing apparatus; and
providing a customized mask pack for the user based on the output data of the user.

12. The method of claim 11, wherein the user terminal or the IoT beauty device generates a third skin measurement information, after the user uses the customized mask pack.

13. The method of claim 11, wherein the user terminal further comprising:
generating a first facial image of the user based on the first and second skin measurement information;
generating a second facial image of the user based on the third skin measurement information; and
comparing and displaying the first facial image and the second facial image.

14. The method of claim 11, wherein the user terminal further comprising:
generating a virtual mask pack based on the skin diagnosis information by receiving the skin diagnosis information from the artificial intelligence server; and
superimposing the fictitious mask pack on the face image of the user to display an output image,
wherein the output image indicates an animated effect of improving skin by the virtual mask pack.

15. The method of claim 11, wherein the AI server further comprising:
forming a plurality of partial face images corresponding to the plurality of sub-regions by dividing the face image of the user into a plurality of sub-regions; and
generating skin diagnostic information for each facial region of the user based on the plurality of partial facial image.
